(19) United States
(12) Patent Application Publication
(10) Pub. No.: US 2026/0013764 A1
(43) Pub. Date: Jan. 15, 2026

(54) DETECTING DEPRESSION CUES IN EEG SIGNALS USING POWER SPECTRAL DENSITY BASED FEATURES FOR DEEP LEARNING

(71) Applicant: Newton Howard, Potomac, MD (US)

(72) Inventor: Newton Howard, Potomac, MD (US)

(21) Appl. No.: 19/266,123

(22) Filed: Jul. 10, 2025

Related U.S. Application Data
(60) Provisional application No. 63/669,979, filed on Jul. 11, 2024.

(51) Int. Cl. A61B 5/16; A61B 5/00; A61B 5/291; A61B 5/31; A61B 5/369; G16H 50/20

(52) U.S. Cl. CPC A61B 5/165; A61B 5/291; A61B 5/31; A61B 5/369; A61B 5/7225; A61B 5/7257; A61B 5/7264; G16H 50/20; A61B 2562/046

(57) ABSTRACT

The present invention relates to techniques for detection of depression cues in EEG signals using power spectral density-based features for deep learning. In an embodiment, a system for detecting depression in a person may comprise an Electroencephalogram (EEG) electrode array attached to the person, the EEG electrode array comprising at least ten electrodes adapted to read EEG signals, an amplifier, a filter, and an Analog to Digital Convert to digitize the read EEG signals, and computing circuitry comprising a processor, memory accessible by the processor, and computer program instructions stored in the memory and executable by the processor to perform: processing on the digitized read EEG signals to detect signal patterns indicative of depression in the person, and outputting a prediction indicating a likelihood of depression in the person based on the deep learning processing.

### CROSS-REFERENCE TO RELATED APPLICATIONS

This claims the benefit of U.S. Provisional Application No. 63/669,379, filed Jul. 10, 2024, the contents of which are incorporated herein in their entirety.

### BACKGROUND

The present invention relates to techniques for detection of depression cues in EEG signals using power spectral density-based features for deep learning.

Depression is a pervasive mental health condition that can significantly impair an individual's emotional and physical wellbeing. Depression can negatively impact the quality of life and increase the risk of chronic diseases. A need arises for early and accurate detection of depression, which is crucial for effective intervention and treatment.

### SUMMARY

Embodiments may relate to techniques for detecting depression using machine learning that is applied to electroencephalogram (EEG) signals. The process begins with using Independent Component Analysis (ICA), which is a powerful technique implemented in MNE (MNE-Python) for removing artefacts from EEG/MEG data. Then, three digital filters (lowpass filter at 80.0 Hz, high pass filter at 0.5 Hz and bandpass between 58.0 Hz and 62.0 Hz) were used to enhance signal quality. Features are extracted from pre-processed EEG signals, focusing on power spectral densities across standard frequency bands. These features serve as inputs to a Convolutional Neural Network (CNN) designed for classifying depressive and non-depressive states.

In an embodiment, a method for detecting depression in a person may comprise attaching an Electroencephalogram (EEG) electrode array to the person, the EEG electrode array comprising at least ten electrodes adapted to read EEG signals, amplifying, filtering, and digitizing the read EEG signals, performing processing on the digitized read EEG signals to detect signal patterns indicative of depression in the person, the processing performed using computing circuitry comprising a processor, memory accessible by the processor, and computer program instructions stored in the memory and executable by the processor to perform the processing, and outputting a prediction indicating a likelihood of depression in the person based on the deep learning processing.

In embodiments, the processing may comprise segmenting the digitized read EEG signals into chunks, each chunk corresponding to a specific scoring period, removing artifacts from each chunk, filtering each chunk, extracting features relating to a power of frequency bands from each chunk, and performing deep learning processing on the extracted features to generate a prediction indicating a likelihood of depression in the person. The deep learning processing may comprise a Convolutional Neural Network (CNN). The feature extraction may comprise performing a Fourier Transform on each chunk to generate a power spectral density (PSD) representation of each chunk, identifying frequency components corresponding to each frequency band, and summing the PSD components to obtain the power for each frequency band.

In an embodiment, a computer program product for generating an encryption key, the computer program product comprising a non-transitory computer readable storage having program instructions embodied therewith, the program instructions executable by a computer, to cause the computer to perform a method that may comprise performing processing on digitized read EEG signals to detect signal patterns indicative of depression in the person, the processing performed using computing circuitry comprising a processor, memory accessible by the processor, and computer program instructions stored in the memory and executable by the processor to perform the processing, the digitized read EEG signals obtained by attaching an Electroencephalogram (EEG) electrode array to the person, the EEG electrode array comprising at least ten electrodes adapted to read EEG signals and amplifying, filtering, and digitizing the read EEG signals, and outputting a prediction indicating a likelihood of depression in the person based on the deep learning processing.

In embodiments, the processing may comprise segmenting the digitized read EEG signals into chunks, each chunk corresponding to a specific scoring period, removing artifacts from each chunk, filtering each chunk, extracting features relating to a power of frequency bands from each chunk, and performing deep learning processing on the extracted features to generate a prediction indicating a likelihood of depression in the person. The deep learning processing may comprise a Convolutional Neural Network (CNN). The feature extraction may comprise performing a Fourier Transform on each chunk to generate a power spectral density (PSD) representation of each chunk, identifying frequency components corresponding to each frequency band; and summing the PSD components to obtain the power for each frequency band.

In an embodiment, a system for detecting depression in a person may comprise an Electroencephalogram (EEG) electrode array attached to the person, the EEG electrode array comprising at least ten electrodes adapted to read EEG signals, an amplifier, a filter, and an Analog to Digital Convert to digitize the read EEG signals, and computing circuitry comprising a processor, memory accessible by the processor, and computer program instructions stored in the memory and executable by the processor to perform processing on the digitized read EEG signals to detect signal patterns indicative of depression in the person, and outputting a prediction indicating a likelihood of depression in the person based on the deep learning processing.

In embodiments, the processing may comprise segmenting the digitized read EEG signals into chunks, each chunk corresponding to a specific scoring period, removing artifacts from each chunk, filtering each chunk, extracting features relating to a power of frequency bands from each chunk, and performing deep learning processing on the extracted features to generate a prediction indicating a likelihood of depression in the person. The deep learning processing may comprise a Convolutional Neural Network (CNN). The feature extraction may comprise performing a Fourier Transform on each chunk to generate a power spectral density (PSD) representation of each chunk, identifying frequency components corresponding to each frequency band, and summing the PSD components to obtain the power for each frequency band.

### BRIEF DESCRIPTION OF THE DRAWINGS

FIG. 1 is an exemplary block diagram of an Electroencephalogram (EEG) system in which embodiments may be implemented.

FIG. 2 illustrates an example of the placement of electrodes for an EEG, in accordance with present embodiments.

FIG. 3 illustrates an example of EEG signals that may be acquired and digitized to form input signals to the computational framework.

FIG. 4 is an exemplary block diagram of the structure of CNNs classifier.

FIG. 5 an example of the accuracy of training as well as validation.

FIG. 6 illustrates an example of the training and validation loss.

FIG. 7 illustrates an example of a Receiver Operating Characteristic (ROC) for model prediction.

FIG. 8 is an exemplary flow diagram of a process for classifying EEG signals to detect depression.

FIG. 9 is an exemplary block diagram of a computer system, in which processes and components involved in the embodiments described herein may be implemented.

### DETAILED DESCRIPTION

Embodiments may relate to techniques for detecting depression using machine learning that is applied to electroencephalogram (EEG) signals. The process begins with using Independent Component Analysis (ICA), which is a powerful technique implemented in MNE (MNE-Python) for removing artefacts from EEG/MEG data. Then, three digital filters (lowpass filter at 80.0 Hz, high pass filter at 0.5 Hz and bandpass between 58.0 Hz and 62.0 Hz) were used to enhance signal quality. Features are extracted from pre-processed EEG signals, focusing on power spectral densities across standard frequency bands. These features serve as inputs to a Convolutional Neural Network (CNN) designed for classifying depressive and non-depressive states. The chosen CNN proves efficient in extracting and processing local patterns in 1D signals. The proposed model has achieved accuracy rates, with 98% accuracy on the training set and 97% on the validation set. Testing on a separate dataset consisting of 10 Major Depressive Disorder (MDD) patients and 12 healthy controls shows the model can correctly identify 70% of MDD cases and 83% of healthy controls. The results indicate the potential of feature based deep learning for the analysis of EEG signals in improving depression diagnosis, providing a useful tool for mental health applications.

Electroencephalogram (EEG) signals are not just data points; they are windows into the intricate electrical activity associated with brain function and disorders. Analysing brain signals typically involves several steps: data acquisition, preprocessing, feature extraction, feature selection, model training and classification, and performance evaluation. The use of digital signal processing and machine learning (ML) techniques in analysing these signals is of significant interest, as it often provides insights into the overall condition of the body and the state of the brain.

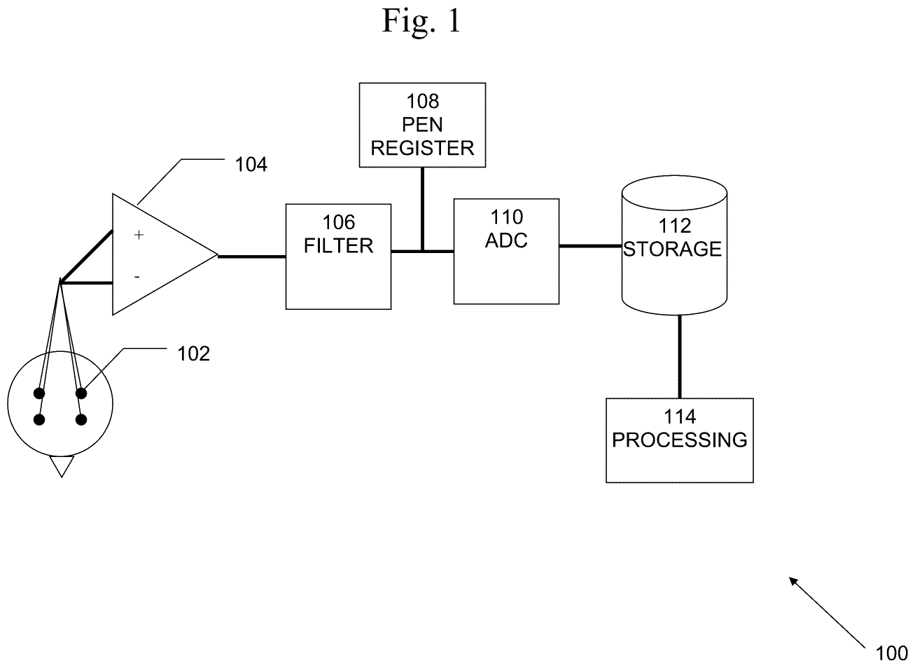

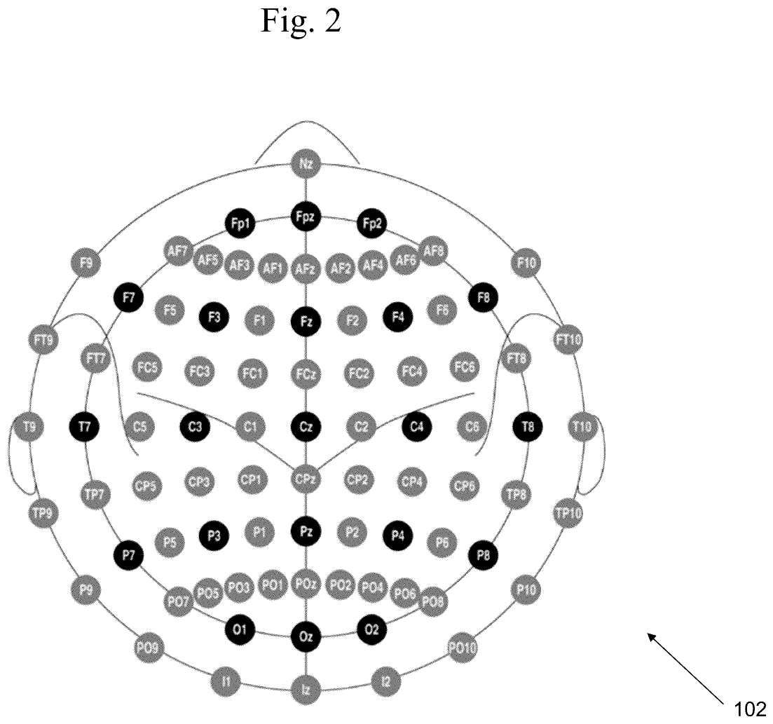

An exemplary Electroencephalogram (EEG) system 100 in which embodiments may be implemented is shown in FIG. 1. This example may include an electrode array 102 arranged on a patient, multiple channels of differential amplifiers 104, multiple channels of filtering 106, pen-type register 108, Analog to Digital Converter (ADC) 110, digital storage 112, and internal or external processing system 114. Electrode array 102 may include a 10-20 electrode placement method, an example of which is shown in FIG. 2. Amplified and filtered EEG signals may be sampled, quantified, and coded to convert them into digital form. Because the effective bandwidth is ˜100 Hz, a minimum sample frequency of 200 Hz (to meet Nyquist criteria) is usually sufficient for sampling the EEG for the majority of applications

There are many applications for EEG signal analysis that allow disease diagnosis to even brain-computer interfaces (BCIs). Epilepsy, a condition extensively examined in EEG signal analysis, is marked by recurrent seizures, and is recognized as a long-term neurological ailment. Although EEG is employed to detect seizure onset and diagnose epilepsy, the existing method is laborious and prone to subjectivity, possibly leading to varying diagnoses among specialists. Consequently, there has been a focused endeavour in academia to introduce technological advancements, like automated seizure detection techniques, to streamline and uniformize diagnostic processes.

EEG signal analysis is additionally connected to the BCI domain, which could be a quickly developing field of inquiry; it is a curious field since it permits a communication bridge between the outside world and the human brain. It has been connected to assistive gadgets which have been utilized to reestablish development to patients, as well as retraining patients to recapture engine usefulness. BCI frameworks work by analysing the approaching brain waves from the EEG and changing the flag into suitable activity. In any case, there are numerous challenges in this space in terms of ease of use, preparation, data exchange rate, and specialized challenges.

Embodiments may include features relating to depression detection using EEG signals, such as:
Feature Extraction Methodology: Emphasis on power spectral densities across standard frequency bands provides a robust set of features that capture the essential characteristics of EEG signals associated with depressive and non-depressive states.
Advanced CNN Architecture: The application of Convolutional Neural Networks (CNNs) designed specifically for 1D signal processing leverages their ability to efficiently extract and process local patterns in EEG signals, leading to high classification accuracy.

### Data Set Description

The data set used in this study was downloaded from the University of Texas, which includes EEG recordings collected from both depressive and non-depressive patients. The University of Texas EEG dataset consists of 64 recordings, including 28 from individuals diagnosed with Major Depressive Disorder (MDD) and 36 from healthy controls (HC). The raw data is provided in three file formats: .eeg, .vhdr, and .vmrk, which were subsequently converted to .bdf files for our experiments. The EEG recordings were sampled at a rate of 500 Hz. We divided the dataset into training and test sets, with the training set containing 18 MDD and 24 HC recordings, and the test set comprising 10 MDD and 12 HC recordings that were not used during training or validation. The training set was further split into 70% for training and 30% for validation purposes. The EEG was recorded using a 64-channel system, covering the major regions of the scalp according to the 10-20 system, including [‘Fp1’, ‘Fz’, ‘F3’, ‘F7’, ‘FT9’, ‘FC5’, ‘FC1’, ‘C3’, ‘T7’, ‘TP9’, ‘CP5’, ‘CP1’, ‘Pz’, ‘P3’, ‘P7’, ‘O1’, ‘Oz’, ‘O2’, ‘P4’, ‘P8’, ‘TP10’, ‘CP6’, ‘CP2’, ‘C4’, ‘T8’, ‘FT10’, ‘FC6’, ‘FC2’, ‘F4’, ‘F8’, ‘Fp2’, ‘AF7’, ‘AF3’, ‘AFz’, ‘F1’, ‘F5’, ‘FT7’, ‘FC3’, ‘FCz’, ‘C1’, ‘C5’, ‘TP7’, ‘CP3’, ‘P1’, ‘P5’, ‘PO7’, ‘PO3’, ‘POZ’, ‘PO4’, ‘PO8’, ‘P6’, ‘P2’, ‘CPz’, ‘CP4’, ‘TP8’, ‘C6’, ‘C2’, ‘FC4’, ‘FT8’, ‘F6’, ‘F2’, ‘AF4’, ‘AF8’, ‘CantherLeft’, ‘VEOGLeft’, ‘VEOGRight’, ‘CantherRight’]. In our work, we have selected 19 channels as Fp2, F8, T8, P8, O2, F3, Fz, F4, C3, C5, C4, P3, Pz, P4, Fp1, F7, T7, P7, and O1.

The data set was divided into training 70% and validation 30% sets, ensuring that the model's performance could be evaluated on unseen data to assess its generalisation capabilities.

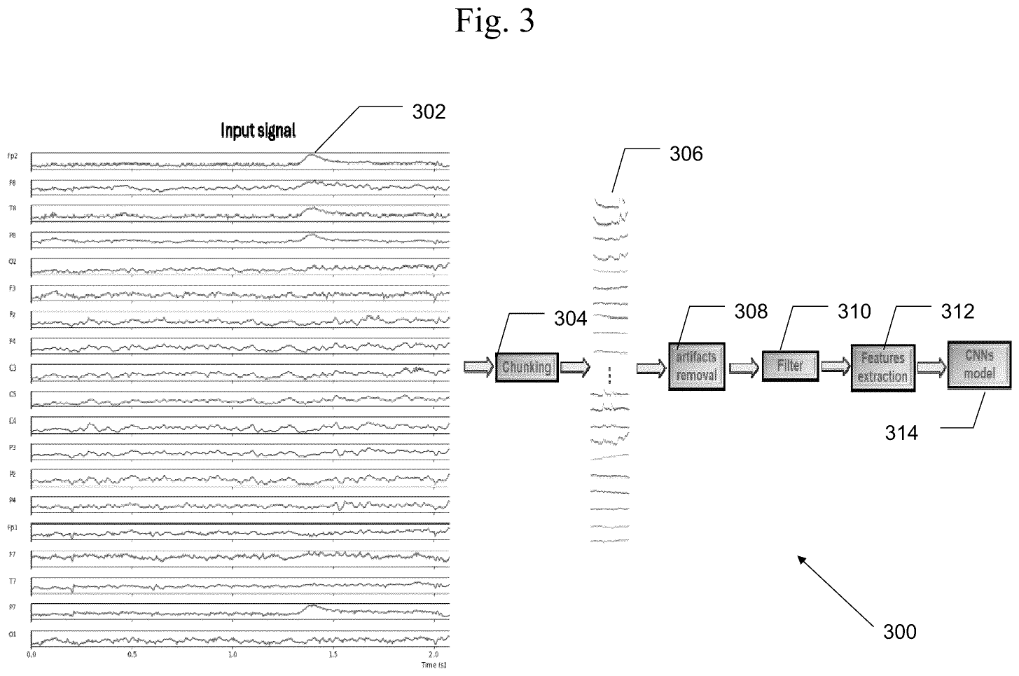

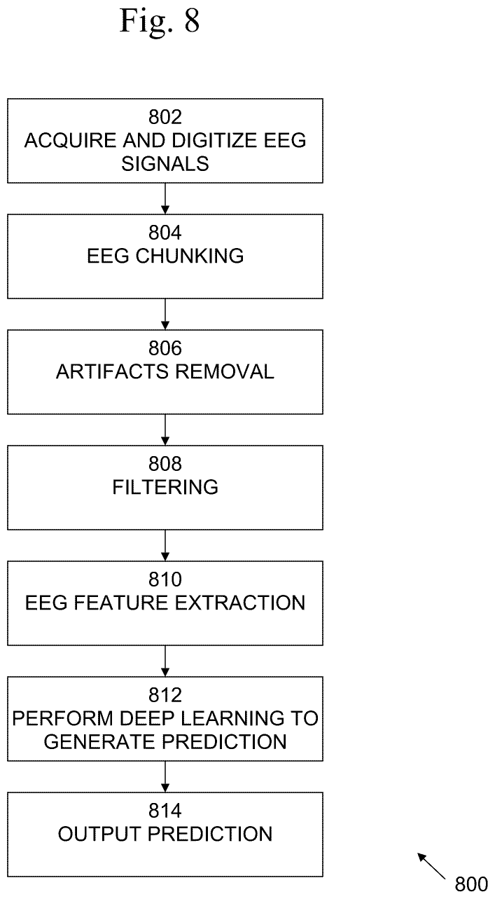

An exemplary flow diagram of a process 800 for classifying EEG signals to detect depression is shown in FIG. 8. involves several critical steps designed to preprocess effectively, chunk the EEG signals into segments, remove artefacts, filter, extract features, and ultimately classify the signals using a Convolutional Neural Network (CNN) model. The comprehensive process is detailed as follows, and is best viewed with reference to FIG. 3, which provides an overview of a computational framework for classifying EEG signals.

Process 800 begins with 802, in which EEG signals may be acquired and digitized to form input signals 302, shown in FIG. 3, to the computational framework. At step 804, EEG Chunking may be performed. The EEG signal is segmented into chunks, each corresponding to a specific scoring period. This segmentation process allows for the analysis of the EEG signal in smaller, more manageable segments 306 while preserving temporal information. The duration of each segment is determined by the scoring period, and the degree of overlap between consecutive segments is controlled by the overlap parameter. Examples of values of these parameters that may be used are ‘scoring_period’: 8.0, ‘scoring_period_overlap’: 0.0 and duration will be scoring_period*sampling rate.

At 806, Artifacts removal 308 may be performed. Artifacts in EEG signals present significant challenges for accurate analysis and interpretation. Employing a combination of detection and removal techniques, tailored to specific artifact types and applications, is often necessary. Advances in computational methods, particularly machine learning, offer promising improvements in the automation and accuracy of artifact management in EEG data, enhancing the reliability and utility of this vital neurophysiological tool. One of the common artifacts is Eye blinking artifacts which is a common source of contamination in EEG signals, particularly affecting frontal electrodes due to their proximity to the eyes. These artifacts occur as a result of the electrical activity generated by the muscles surrounding the eyes during blinking. The characteristics of eye blinking artifacts typically involve sharp transients or large, slow waves in the EEG signal.

To process and correct the artifacts, there are many strategies that could use to remove artifacts from the signal. However, there are two steps, first, we have to identify if the signal has artifacts or not by computing the sort of features that are related to the abnormal cases. Those features are Kurtosis (K) and Relative Roughness (RR) that those values should be as mentioned below.

If K>=Threshold_k or RR>=Threshol_rr then there is artifact. Where Threshold k=4.0 and Threshold_rr=0.3 after many experiments.

Once the artifacts are detected, next will be the correction phase by applying for example, the FastICA algorithm implemented in the MNE-Python library. FastICA is chosen for its efficiency and robustness in dealing with large EEG datasets.

At 808, Filtering 310 may be performed. After artefact removal, the EEG segments are further processed using three types of digital filters: low-pass, high-pass, and band-stop filters. These filters are crucial in isolating the frequency components of interest and removing unwanted noise, thereby enhancing the quality of the EEG data for subsequent feature extraction and analysis.

Low-Pass Filter: A low-pass filter (with cutoff Frequency 80.0 Hz and second-order by default) is designed to allow frequencies below a certain cutoff frequency to pass through while attenuating higher frequencies. This filter is useful for removing high-frequency noise that can obscure the true brain activity signals. Mathematically, a low-pass filter can be represented using a Butterworth filter design, which is characterized by its flat frequency response in the passband.

High-Pass Filter: A high-pass filter (with cutoff Frequency 0.5 Hz and second-order by default), conversely, allows frequencies above a certain cutoff frequency to pass through while attenuating lower frequencies. This filter is useful for removing low-frequency drift and baseline wander, which are common in EEG recordings.

Band-Stop Filter: Also known as a notch filter (Lower Cutoff Frequency 58.0 Hz and Upper Cutoff Frequency 62.0 Hz and second-order by default), a band-stop filter attenuates frequencies within a specific range while allowing frequencies outside this range to pass through. This filter is particularly useful for removing power line noise (typically at 50 Hz or 60 Hz), which can interfere with EEG signal analysis.

These filters may be implemented using the Butterworth design due to their desirable properties, such as a maximally flat response in the passband and a smooth roll-off around the cutoff frequencies. By applying these filters to the EEG segments, we ensure that only the relevant frequency components are retained, leading to cleaner and more interpretable signals. This preprocessing step significantly enhances the quality of the input data for subsequent feature extraction and classification tasks.

At 810, EEG Feature Extraction 312 may be performed. Feature extraction follows signal preprocessing and is a crucial aspect of analysing biomedical signals. With the rise of big data, particularly in medical contexts like EEG signal acquisition with its multi-hour and multi-channel nature, feature extraction reduces dimensionality and compresses data. This allows for representing data with a smaller feature subset, optimising the utilisation of machine learning and artificial intelligence techniques for tasks such as classification and diagnosis. It's important to note that not all features are relevant for specific applications; the “useful” ones should ideally accurately represent the underlying signal.

Moreover, Alotaiby et al. and Krishnan mentioned that it is important to note that EEG signals carry properties that complicate the feature extraction and signal analysis process. EEG signals are non-stationary, non-linear, non-Gaussian, and non-short form. In order to build a robust process, those kinds of properties must be considered to extract features.

EEG signals are composed of oscillatory patterns across different frequency bands, each reflecting distinct aspects of neural processing. These frequency bands entail breaking down the EEG signal into individual frequency components, each exhibiting distinct functional properties. The standard frequency bands and their approximate spectral ranges are Delta (0-4 Hz), Theta (4-8 Hz), Alpha (8-16 Hz), Beta (16-32 Hz), and Gamma (32-80 Hz). These bands allow us to capture significant neurophysiological indicators pertinent to depression.

We obtained BDF files containing EEG data from individuals diagnosed with depression and non-depression. These files were recorded using a BioSemi EEG system with a standard electrode montage. BDF, a 24-bit iteration derived from the widely used 16-bit EDF format utilized in earlier BioSemi models equipped with 16-bit converters, closely mirrors EDF. While EDF initially found prominence in sleep research, the BDF/EDF combination swiftly garners attention across various EEG applications, alongside ECG body surface potential mapping and EMG.

The EEG data were sampled at a frequency of 500 Hz and consisted of signals recorded from multiple EEG channels, including Fp2, F8, T8, P8, O2, F3, Fz, F4, C3, C5, C4, P3, Pz, P4, Fp1, F7, T7, P7, and O1.

Our feature extraction methodology involves the following steps:

### EEG Signal Segmentation:

We divide the EEG signal into overlapping segments or chunks, each corresponding to a specified scoring period. The scoring period determines the duration of each segment, while the overlap parameter controls the degree of overlap between consecutive segments.

### Power Spectral Density Calculation

Perform FFT on the pre-processed signal to convert it from the time domain to the frequency domain, resulting in a power spectral density (PSD) representation of the signal. Next, identify the frequency components corresponding to each band: Delta (0.5-4 Hz), Theta (4-8 Hz), Alpha (8-12 Hz), Beta (12-30 Hz), and Gamma (30-50 Hz). Sum the PSD values within these ranges to obtain the power for each band, providing insights into the signal's frequency composition and underlying neural activity. This method is fundamental in neuroscience for analysing brain function and diagnosing neurological conditions. The steps below can express the computations of features extraction:
Perform FFT (Fast Fourier Transform) on the pre-processed signal to convert it from the time domain to the frequency domain.

$$X ( f ) = FFT ( x ( t ) ) \qquad ( 1 )$$

where x(t) is pre-processed signal in the time domain and X(f) in frequency domain after applying FFT.

Calculate the Power Spectral Density (PSD) from the FFT result.

$$S ( f ) = {\vert X ( f ) \vert}^{2} \qquad ( 2 )$$

where S(f) represents the PSD of the signal at frequency f.

Define frequency bands using frequencies mentioned above:

$${P}_{D e l t a} = {\int }_{05 Hz}^{4 Hz} S ( f ) df \qquad ( 3 )$$

$${P}_{Theta} = {\int }_{4 Hz}^{8 Hz} S ( f ) df \qquad ( 4 )$$

$${P}_{Alpha} = {\int }_{8 Hz}^{12 Hz} S ( f ) df \qquad ( 5 )$$

$${P}_{B e t a} = {\int }_{12 Hz}^{30 Hz} S ( f ) df \qquad ( 6 )$$

$${P}_{G a m m a} = {\int }_{30 Hz}^{50 Hz} S ( f ) df \qquad ( 7 )$$

After extracting features within predefined frequency bands (Delta, Theta, Alpha, Beta, and Gamma) from each chunk, we will have a total of 19 EEG channels *5 bands. These features represent the power within each frequency band for each channel. These features were organised into an array to be suitable for machine learning.

At 812, Deep learning 314 may be performed and a prediction of a likelihood that the patient is depressed may be generated. At 814, the generated prediction may be output. Convolutional Neural Networks CNNs architecture is a type of deep learning which train using a huge, labelled data set to archive specific task such as images.

Embodiments may include deep learning, such as Convolutional Neural Networks (CNNs). Firstly, CNNs have a unique advantage in automatically learning hierarchical feature representations from raw data. For EEG signals. Secondly, EEG data, comprising multiple frequency bands (5 bands) and (19) channels, is inherently multidimensional. CNNs are well-suited to process such data due to their ability to handle multiple input dimensions and extract spatial and temporal features effectively. There are numerous studies have demonstrated that CNNs achieve state-of-the-art results in various domains including biomedical signal processing.

There are many types of CNN architectures, including 1D and 2D structures. In this project, we defined our model with three 1D convolutional layers. The decision to use 1D-CNN instead of dense DNN, LSTM, or BLSTM models was based on some factors like; 1D-CNNs are particularly effective for sequential data, such as time series or signal data, where they can efficiently capture local patterns and dependencies. Recent studies have demonstrated the effectiveness of 1D-CNNs in processing EEG signals. For instance, a study by Clevert et al. highlighted that 1D-CNNs could efficiently handle the temporal dynamics of EEG data for motor imagery classification. Another study utilized a 1D-CNN for EEG-based emotion recognition, showcasing its ability to capture intricate patterns and relationships within the data (MDPI). Additionally, while CNNs are commonly associated with 2D data like images due to their ability to apply pooling operations and extract hierarchical features, 1D-CNNs also benefit from pooling mechanisms.

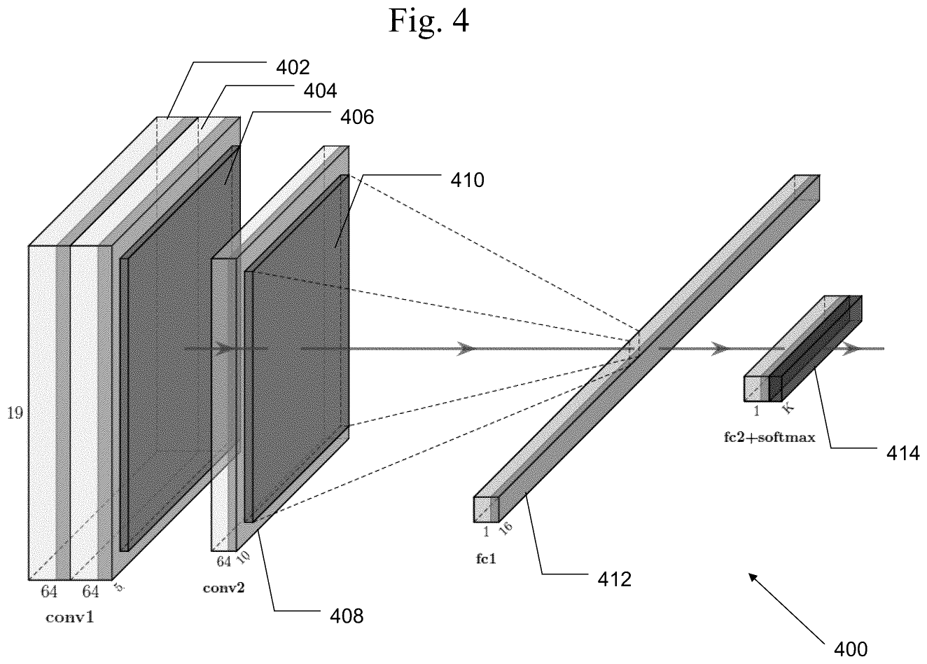

An exemplary block diagram of the structure of CNNs classifier 400 is shown in FIG. 4. In the first layer 402; there may be 64 filters with size 3 and the shape of the input may be (19, 5). Then, the second layer 404 is conv1d_1 which may have the same structure as the first layer followed by a pooling layer 406, which downsamples the input representation by taking the maximum value over a spatial window of size pool_size. The output from the previous layer will be input to the third layer 408 followed by MaxPooling1D 410 to down sample the input representation.

Finally, there will be a flatten layer 412, which flattens the learned features to be a dimension vector and pass them through a fully connected layer 414 prior to the output layer being used to do a prediction (normal or abnormal). The idea beyond using a fully connected layer is to provide a buffer between the features that have been learned previously and the output to interpret the learned features before making a prediction.

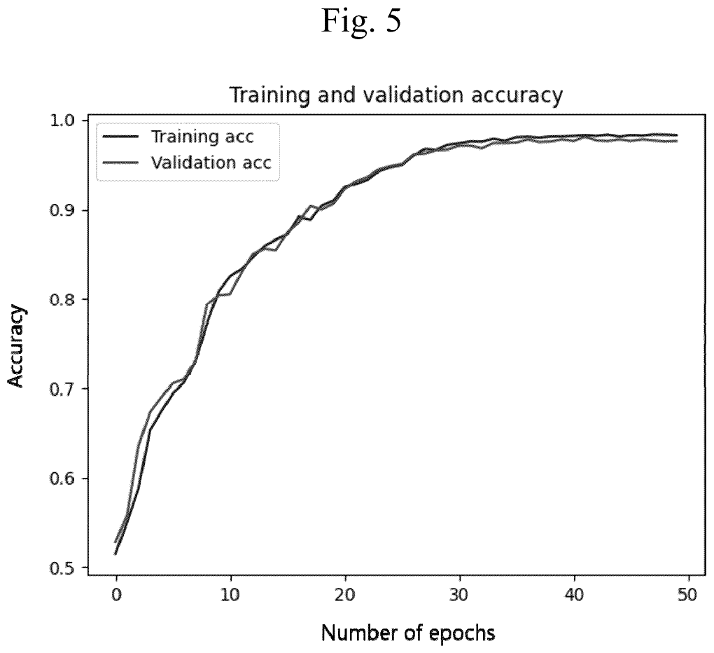

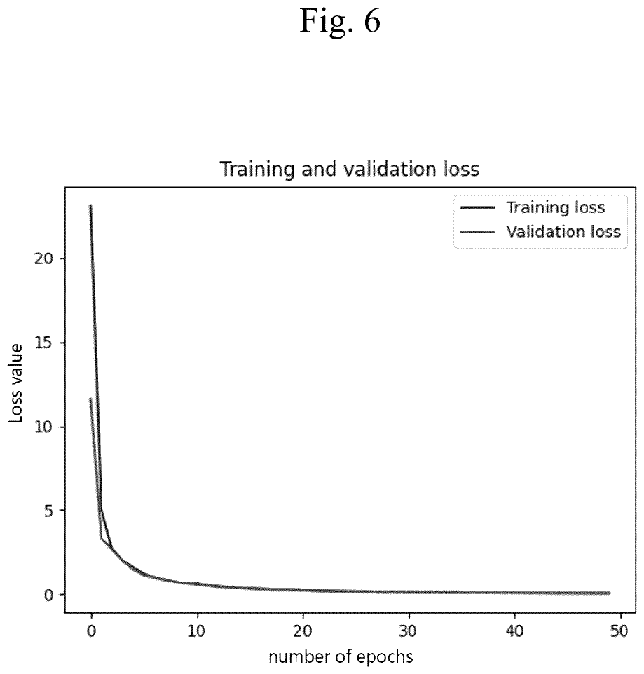

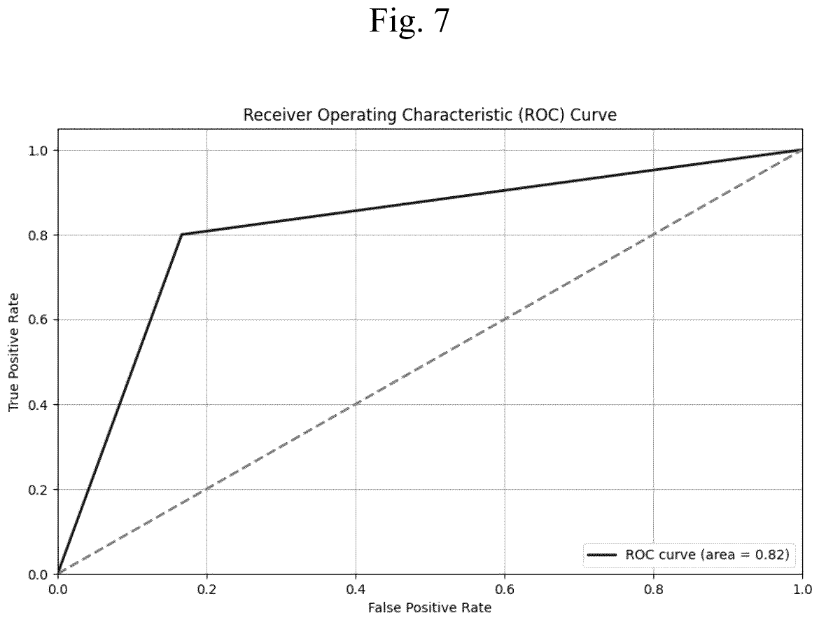

Before being used, the deep learning model may be trained. During the training stage, the model will establish the optimum way for mapping input samples of the data to particular class labels by using the training dataset. To train a model, we split the data into two sets; training 70% (to train the model) and validation 30% (to evaluate model accuracy). Another step for training, we have to adjust set of Hyperparameters (epochs-learning rate-activation function) before fitting training features to the model. In our case, for learning rate the beast value was 0.001, batch size was 32, and epoch=50 and the training accuracy was around 98% and validation about 97%. FIGS. 6 and 7 show the conclusion of the model training phase. FIG. 5 illustrates an example of the accuracy of training as well as validation. In addition, in FIG. 6 illustrates an example of the training and validation loss.

To further assess the model's accuracy, we conducted tests using two distinct groups: patients diagnosed with Major Depressive Disorder (MDD) and Healthy Controls (HC), which we have not used in anywhere during model training and validation. Our test set included 10 MDD patients and 12 HC individuals. The results indicated that the model correctly identified 70% of the MDD cases and 83% of the HC cases, achieving an overall accuracy of approximately 86.36%. The precision for MDD was approximately 81.82%, indicating the proportion of correctly identified MDD cases among all predicted MDD cases. Moreover, the model's recall, measuring its ability to identify all actual MDD cases, stood at 90%. The F1 Score, which combines precision and recall into a single metric, was approximately 85.71%.

In addition, we plotted the Receiver Operating Characteristic (ROC) curve for the model by evaluating the test data to distinguish between MDD and HC.

FIG. 7 illustrates an example of a Receiver Operating Characteristic (ROC) for model prediction. The ROC curve in FIG. 7, represented by the solid line, plots the True Positive Rate (TPR) against the False Positive Rate (FPR), illustrating the model's performance. The curve starts at the origin (0,0), rises sharply, and then levels off, indicating that the model initially achieves a high TPR with a low FPR before the rate of increase in FPR becomes more gradual. The Area Under the Curve (AUC) is 0.82, suggesting that the CNN model has good discriminative power in differentiating between MDD patients and HC, significantly better than random guessing (represented by the dashed line with an AUC of 0.5). This evaluation demonstrates the model's effectiveness in accurately classifying individuals into the MDD or HC groups based on the test data.

The results of our study highlight the efficacy of deep learning techniques, particularly Convolutional Neural Networks (CNNs), in the classification of depression using EEG signals. By leveraging the unique capabilities of CNNs to learn intricate patterns in the data, we achieved notable accuracy and reliability in distinguishing depressive states from non-depressive ones.

The preprocessing steps, including artifact removal using a GAN-based autoencoder and the application of various digital filters, were crucial in enhancing the signal quality. These steps addressed common EEG signal challenges, such as noise and artifacts, which can significantly impair analysis. The robust preprocessing pipeline ensured that the subsequent feature extraction and classification stages operated on clean, high-quality data.

Feature extraction focused on power spectral densities across different frequency bands (Delta, Theta, Alpha, Beta, and Gamma) proved effective in capturing relevant neurophysiological indicators of depression. This approach aligns with established neuroscience research, which links specific EEG frequency bands to various cognitive and emotional states.

Our CNN model, meticulously designed with multiple convolutional layers and pooling operations, exhibited robust performance metrics throughout both the training and validation phases. The model achieved an impressive training accuracy of 98% and a validation accuracy of 97%. These high accuracy rates affirm the model's capacity to generalize effectively to unseen data, a crucial requirement for real-world clinical applications.

However, several limitations and future directions warrant discussion. The dataset, although robust, included a limited number of participants. Expanding the dataset size and diversity could further enhance the model's generalizability.

In this paper, we have discussed an approach to analysis depression using deep learning techniques applied to EEG signals. Through meticulous preprocessing, feature extraction, and classification, our proposed methodology highlights the potential of EEG-based deep learning models in mental health diagnostics. The use of Independent Component Analysis (ICA) and digital filtering significantly improved the signal quality by effectively removing artifacts. Our feature extraction strategy, focusing on power spectral densities across standard frequency bands, provided a robust representation of the underlying neural activity associated with depressive and non-depressive states.

The Convolutional Neural Network (CNN) architecture utilized in this study leveraged the strengths of CNNs in processing 1D signals, achieving high accuracy rates of 98% on the training set and 97% on the validation set. Testing on an independent dataset revealed that the model correctly identified 70% of MDD cases and 83% of healthy controls, underscoring its practical applicability in clinical settings.

These results underscore the promising utility of deep learning techniques in depression detection through EEG signal analysis. The successful application of deep learning to EEG signals for depression detection opens new avenues for non-invasive and efficient mental health diagnostics. This approach offers a promising tool for early intervention and treatment, potentially improving patient outcomes. However, further refinement and validation with larger and more diverse datasets are necessary to enhance the model's diagnostic capabilities and ensure its applicability in real-world clinical settings. Overall, our study highlights the potential of deep learning as a valuable tool in mental health diagnosis and underscores the importance of continued research in this area.

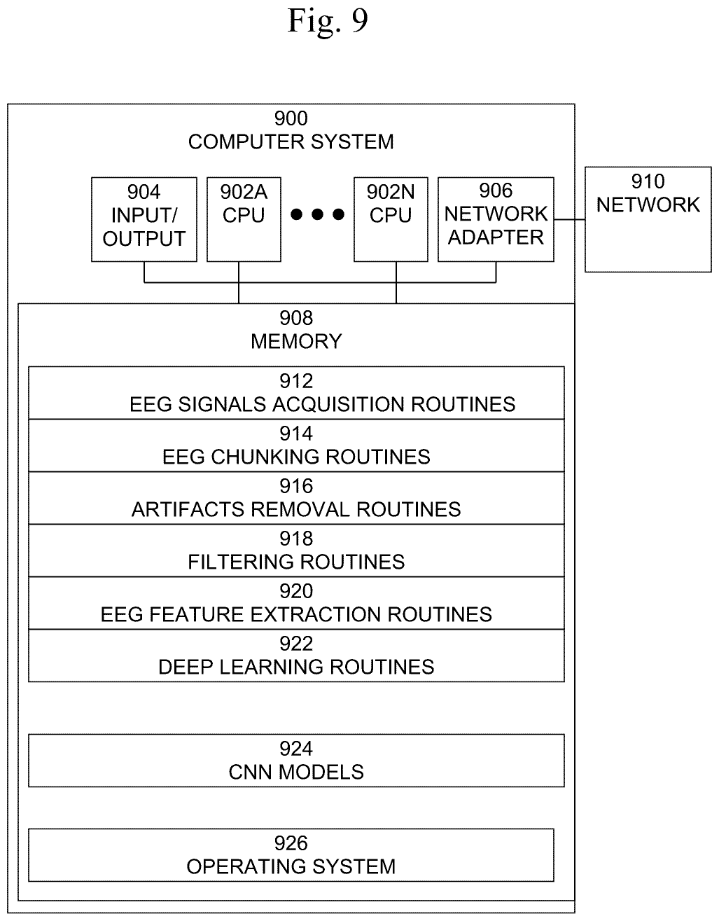

An exemplary block diagram of a computer system 900, in which processes and components involved in the embodiments described herein may be implemented, is shown in FIG. 9. Computer system 900 may be implemented using one or more programmed general-purpose computer systems, such as embedded processors, systems on a chip, personal computers, workstations, server systems, and minicomputers or mainframe computers, or in distributed, networked computing environments. Computer system 900 may include one or more processors (CPUs) 902A-902N, input/output circuitry 904, network adapter 906, and memory 908. CPUs 902A-902N execute program instructions in order to carry out the functions of the present communications systems and methods. Typically, CPUs 902A-902N are one or more microprocessors, such as an INTEL CORE® processor. FIG. 9 illustrates an embodiment in which computer system 900 is implemented as a single multi-processor computer system, in which multiple processors 902A-902N share system resources, such as memory 908, input/output circuitry 904, and network adapter 906. However, the present communications systems and methods also include embodiments in which computer system 900 is implemented as a plurality of networked computer systems, which may be single-processor computer systems, multi-processor computer systems, or a mix thereof.

Input/output circuitry 904 provides the capability to input data to, or output data from, computer system 900. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 906 interfaces device 900 with a network 910. Network 910 may be any public or proprietary LAN or WAN, including, but not limited to the Internet.

Memory 908 stores program instructions that are executed by, and data that are used and processed by, CPU 902 to perform the functions of computer system 900. Memory 908 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 908 may vary depending upon the function that computer system 900 is programmed to perform. In the example shown in FIG. 9, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present systems and methods may include any and all such arrangements.

In the example shown in FIG. 9, memory 908 may include EEG signals acquisition routines 912, EEG chunking routines 914, artifacts removal routines 916, filtering routines 918, EEG feature extraction routines 920, deep learning routines 922, CNN models 924 and operating system 926. Neural signal reading routines 912 may include software to read neural signals, as described above. Neural signal stimulation routines 914 may include software to apply stimulation signals to neural tissue to affect neural activity, as described above. Neural signal analysis routines 916 may include software to analyze read neural signals, as described above. Encryption key generation routines 918 may include software to use analyzed neural signals to generate encryption keys, as described above. Encryption routines 920 may include software to perform speaker-specific detection of a mental disorder, as described above. Operating system 922 may provide overall system functionality.

As shown in FIG. 9, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for detecting depression in a person comprising: attaching an Electroencephalogram (EEG) electrode array to the person, the EEG electrode array comprising at least ten electrodes adapted to read EEG signals;
amplifying, filtering, and digitizing the read EEG signals;
performing processing on the digitized read EEG signals to detect signal patterns indicative of depression in the person, the processing performed using computing circuitry comprising a processor, memory accessible by the processor, and computer program instructions stored in the memory and executable by the processor to perform the processing; and
outputting a prediction indicating a likelihood of depression in the person based on the deep learning processing.

2. The method of claim 1, wherein the processing comprises:
segmenting the digitized read EEG signals into chunks, each chunk corresponding to a specific scoring period;
removing artifacts from each chunk;
filtering each chunk;
extracting features relating to a power of frequency bands from each chunk; and
performing deep learning processing on the extracted features to generate a prediction indicating a likelihood of depression in the person.

3. The method of claim 2, wherein the deep learning processing comprises a Convolutional Neural Network (CNN).

4. The method of claim 2, wherein the feature extraction comprises:
performing a Fourier Transform on each chunk to generate a power spectral density (PSD) representation of each chunk;
identifying frequency components corresponding to each frequency band; and
summing the PSD components to obtain the power for each frequency band.

5. A computer program product for generating an encryption key, the computer program product comprising a non-transitory computer readable storage having program instructions embodied therewith, the program instructions executable by a computer, to cause the computer to perform a method comprising:
performing processing on digitized read EEG signals to detect signal patterns indicative of depression in the person, the processing performed using computing circuitry comprising a processor, memory accessible by the processor, and computer program instructions stored in the memory and executable by the processor to perform the processing, the digitized read EEG signals obtained by attaching an Electroencephalogram (EEG) electrode array to the person, the EEG electrode array comprising at least ten electrodes adapted to read EEG signals and amplifying, filtering, and digitizing the read EEG signals; and
outputting a prediction indicating a likelihood of depression in the person based on the deep learning processing.

6. The computer program product of claim 5, wherein the processing comprises:
segmenting the digitized read EEG signals into chunks, each chunk corresponding to a specific scoring period;
removing artifacts from each chunk;
filtering each chunk;
extracting features relating to a power of frequency bands from each chunk; and
performing deep learning processing on the extracted features to generate a prediction indicating a likelihood of depression in the person.

7. The computer program product of claim 6, wherein the deep learning processing comprises a Convolutional Neural Network (CNN).

8. The computer program product of claim 6, wherein the feature extraction comprises:
performing a Fourier Transform on each chunk to generate a power spectral density (PSD) representation of each chunk;
identifying frequency components corresponding to each frequency band; and
summing the PSD components to obtain the power for each frequency band.

9. A system for detecting depression in a person comprising:
an Electroencephalogram (EEG) electrode array attached to the person, the EEG electrode array comprising at least ten electrodes adapted to read EEG signals;
an amplifier, a filter, and an Analog to Digital Convert to digitize the read EEG signals; and
computing circuitry comprising a processor, memory accessible by the processor, and computer program instructions stored in the memory and executable by the processor to perform:
processing on the digitized read EEG signals to detect signal patterns indicative of depression in the person, and
outputting a prediction indicating a likelihood of depression in the person based on the deep learning processing.

10. The system of claim 9, wherein the processing comprises:
segmenting the digitized read EEG signals into chunks, each chunk corresponding to a specific scoring period;
removing artifacts from each chunk;
filtering each chunk;
extracting features relating to a power of frequency bands from each chunk; and
performing deep learning processing on the extracted features to generate a prediction indicating a likelihood of depression in the person.

11. The system of claim 10, wherein the deep learning processing comprises a Convolutional Neural Network (CNN).

12. The system of claim 10, wherein the feature extraction comprises:
performing a Fourier Transform on each chunk to generate a power spectral density (PSD) representation of each chunk;
identifying frequency components corresponding to each frequency band; and
summing the PSD components to obtain the power for each frequency band.
